SEQUENCE LISTING (68 sequences)

SEQ ID NO: 1 - protein, 7 aa, Unknown
  peptide aptamer
LAREPTS

SEQ ID NO: 2 - protein, 7 aa, Unknown
  peptide aptamer
HELVRSP

SEQ ID NO: 3 - protein, 7 aa, Unknown
  peptide aptamer
WALDRGA

SEQ ID NO: 4 - protein, 7 aa, Unknown
  peptide aptamer
APSTPTP

SEQ ID NO: 5 - protein, 7 aa, Unknown
  peptide aptamer
SMPQTAG

SEQ ID NO: 6 - protein, 7 aa, Unknown
  peptide aptamer
EPLQLKM

SEQ ID NO: 7 - protein, 7 aa, Unknown
  peptide aptamer
ATPLWLK

SEQ ID NO: 8 - protein, 7 aa, Unknown
  peptide aptamer
AKIDART

SEQ ID NO: 9 - protein, 7 aa, Unknown
  peptide aptamer
YNHQRPP

SEQ ID NO: 10 - protein, 7 aa, Unknown
  peptide aptamer
ALIPKPR

SEQ ID NO: 11 - protein, 7 aa, Unknown
  peptide aptamer
QLITKPL

SEQ ID NO: 12 - protein, 7 aa, Unknown
  peptide aptamer
STPIQQP

SEQ ID NO: 13 - protein, 7 aa, Unknown
  peptide aptamer
TFAKSAY

SEQ ID NO: 14 - protein, 7 aa, Unknown
  peptide aptamer
STFTKSP

SEQ ID NO: 15 - protein, 7 aa, Unknown
  peptide aptamer
TPAHNDY

SEQ ID NO: 16 - protein, 7 aa, Unknown
  peptide aptamer
SLSLIQT

SEQ ID NO: 17 - protein, 7 aa, Unknown
  peptide aptamer
NQDVPLF

SEQ ID NO: 18 - protein, 7 aa, Unknown
  peptide aptamer
GPHLGLK

SEQ ID NO: 19 - protein, 7 aa, Unknown
  peptide aptamer
LAGPQMH

SEQ ID NO: 20 - protein, 7 aa, Unknown
  peptide aptamer
SQASSLK

SEQ ID NO: 21 - protein, 7 aa, Unknown
  peptide aptamer
SDLSSPY

SEQ ID NO: 22 - protein, 7 aa, Unknown
  peptide aptamer
LVTTWPA

SEQ ID NO: 23 - protein, 7 aa, Unknown
  peptide aptamer
ALYKNTS

SEQ ID NO: 24 - protein, 7 aa, Unknown
  peptide aptamer
QQISTQM

SEQ ID NO: 25 - protein, 7 aa, Unknown
  peptide aptamer
AWLPAGA

SEQ ID NO: 26 - protein, 7 aa, Unknown
  peptide aptamer
GTWLSRG

SEQ ID NO: 27 - protein, 7 aa, Unknown
  peptide aptamer
GHQVSRL

SEQ ID NO: 28 - protein, 7 aa, Unknown
  peptide aptamer
GPMLARG

SEQ ID NO: 29 - protein, 7 aa, Unknown
  peptide aptamer
TASLVRP

SEQ ID NO: 30 - protein, 7 aa, Unknown
  peptide aptamer
TDSLRLL

SEQ ID NO: 31 - protein, 7 aa, Unknown
  peptide aptamer
AKPDWRF

SEQ ID NO: 32 - protein, 7 aa, Unknown
  peptide aptamer
VQTYARV

SEQ ID NO: 33 - protein, 7 aa, Unknown
  peptide aptamer
YTEPVRE

SEQ ID NO: 34 - protein, 7 aa, Unknown
  peptide aptamer
MSLQQEH

SEQ ID NO: 35 - protein, 6 aa, Unknown
  peptide aptamer
MKWQTV

SEQ ID NO: 36 - protein, 7 aa, Unknown
  peptide aptamer
NERALTL

SEQ ID NO: 37 - protein, 7 aa, Unknown
  peptide aptamer
SPSTHWK

SEQ ID NO: 38 - protein, 7 aa, Unknown
  peptide aptamer
WSTTNVP

SEQ ID NO: 39 - protein, 7 aa, Unknown
  peptide aptamer
YHKLDPL

SEQ ID NO: 40 - protein, 7 aa, Unknown
  peptide aptamer
LSNNNLR

SEQ ID NO: 41 - protein, 7 aa, Unknown
  peptide aptamer
LPAGRVL

SEQ ID NO: 42 - protein, 7 aa, Unknown
  peptide aptamer
STSFWIT

SEQ ID NO: 43 - protein, 7 aa, Unknown
  peptide aptamer
LGSPMSN

SEQ ID NO: 44 - protein, 7 aa, Unknown
  peptide aptamer
WELRPRT

SEQ ID NO: 45 - protein, 7 aa, Unknown
  peptide aptamer
TNIESLK

SEQ ID NO: 46 - protein, 7 aa, Unknown
  peptide aptamer
LYAEVIR

SEQ ID NO: 47 - protein, 7 aa, Unknown
  peptide aptamer
NHSTQMY

SEQ ID NO: 48 - protein, 7 aa, Unknown
  peptide aptamer
NLQIYAV

SEQ ID NO: 49 - protein, 7 aa, Unknown
  peptide aptamer
YDTSSAS

SEQ ID NO: 50 - protein, 7 aa, Unknown
  peptide aptamer
MHSNTWD

SEQ ID NO: 51 - protein, 7 aa, Unknown
  peptide aptamer
TPTTVSY

SEQ ID NO: 52 - protein, 7 aa, Unknown
  peptide aptamer
TSHSILQ

SEQ ID NO: 53 - protein, 12 aa, Unknown
  peptide aptamer
TLRVPPNPNMNV

SEQ ID NO: 54 - protein, 12 aa, Unknown
  peptide aptamer
HVKLVAVADLMN

SEQ ID NO: 55 - protein, 12 aa, Unknown
  peptide aptamer
YHPNGMNPYTKA

SEQ ID NO: 56 - protein, 12 aa, Unknown
  peptide aptamer
EYSPKLFPPHRL

SEQ ID NO: 57 - protein, 12 aa, Unknown
  peptide aptamer
HHLTHANSLTNT

SEQ ID NO: 58 - protein, 12 aa, Unknown
  peptide aptamer
WHWGLLYPASAN

SEQ ID NO: 59 - protein, 12 aa, Unknown
  peptide aptamer
KPLMTYKVIHYV

SEQ ID NO: 60 - protein, 12 aa, Unknown
  peptide aptamer
QPDLSHPSTNAY

SEQ ID NO: 61 - protein, 12 aa, Unknown
  peptide aptamer
FPTNLATRSAMV

SEQ ID NO: 62 - protein, 9 aa, Unknown
  peptide aptamer
LAGPQMHGK

SEQ ID NO: 63 - protein, 12 aa, Unknown
  peptide aptamer
PLRPKSEYPFHY

SEQ ID NO: 64 - protein, 12 aa, Unknown
  peptide aptamer
SYSPKLFPPHRL

SEQ ID NO: 65 - protein, 8 aa, Unknown
  peptide aptamer
SDLSSPYG

SEQ ID NO: 66 - protein, 9 aa, Unknown
  peptide aptamer
SDLSSPYGG

SEQ ID NO: 67 - protein, 13 aa, Unknown
  peptide aptamer
SDLSSPYGGCVVV

SEQ ID NO: 68 - protein, 14 aa, unknown
  peptide aptamer
EYSPKLFPPHRLGK

(19) United States
(12) Patent Application Publication
(10) Pub. No.: US 2017/0349546 A1
(43) Pub. Date: Dec. 7, 2017

(54) Light-Activated Two-Component Protein Binding Matrix

(71) Applicant: National University of Singapore, Singapore (SG)

(72) Inventor: Chester Lee Drum, Singapore (SG)

(21) Appl. No.: 15/521,048

(22) PCT Filed: Oct. 29, 2015
(86) PCT No.: PCT/SG2015/050421

§ 371 Date: Apr. 21, 2017

Related U.S. Application Data
(60) Provisional application No. 62/072,727, filed on Oct. 30, 2014.

(51) Int. Cl. C07D 209/12; C07K 7/08; C07K 7/06; C07K 1/22; C07D 491/107; G01N 33/547; C07D 209/18

(52) U.S. Cl. CPC C07D 209/12; G01N 33/547; C07K 7/08; C07D 209/18; C07K 1/22; C07D 491/107; C07K 7/06

(57) ABSTRACT

A binding pair for chromatographic separation or purification of a molecule of interest, where one binding member of the pair is an isomerizable organic molecule and the other binding member of the pair is an isomer-specific affinity agent bound to a molecule of interest. The binding pair associates and disassociates upon exposure to a binding agent, such as using light, decreased intensity of light, darkness, heat, stress, ions, an isomerizable affinity agent, change in pH, or a combination thereof.

### RELATED APPLICATION

This application claims the benefit of U.S. Provisional Application No. 62/072,727, filed on Oct. 30, 2014. The entire teachings of the above application are incorporated herein by reference.

### BACKGROUND OF THE INVENTION

Effective analytical and preparative tools are necessary for high-throughput resolution, characterization of complex protein mixtures, and for the purification of proteins of quantity, scale, and cost appropriate for commercial exploitation. Liquid chromatography is an important tool in proteomics and large-scale protein purification, allowing the separation of proteins by different properties. Nevertheless, there is a need for chromatographic tools that allow for more efficient protein purification, in a cost effective manner.

### SUMMARY OF THE INVENTION

The invention relates to the discovery of a two-component system for controllably and selectively binding and releasing a molecule of interest. The invention takes advantage of isomerizable organic molecules (a first component in the two-component system) that have different isomeric forms, where in the first isomeric form the isomerizable organic molecule will bind to a binding partner (a second component in the two-component system) and where in a second isomeric form the binding partner will release or dissociate therefrom. The change between isomeric forms is induced by conditions such as light, temperature, pH, the isomer-specific agent or combination of these, and as such the two-component system assembles and disassembles according to the inducing conditions. The two-component system uses an isomerizable organic molecule and its affinity binding partner to separate and/or purify recombinant proteins, for example, in a chromatographic column.

In a first aspect, the invention is a binding pair comprising a first binding member being an isomerizable organic molecule (e.g., photoisomerizable organic molecule), and a second binding member being an isomer-specific affinity agent, wherein the isomerizable organic molecule has a binding affinity for the second binding member under a first condition, and a different binding affinity for the second binding member under a second condition. In an example embodiment of the first aspect of the invention, difference in the binding affinity is such that the first binding member and the second binding member associate under a first condition and disassociate under a second condition. In another example embodiment, the isomer-specific affinity agent comprises a peptide aptamer or a nucleic acid aptamer. In another example embodiment, the isomer-specific affinity agent further comprises a linker that is covalently bound to the peptide aptamer or the nucleic acid aptamer, wherein the linker is capable of non-covalently or covalently binding a molecule of interest. In another example embodiment, the linker comprises a protease sensitive cleavage site. In another example embodiment, the peptide aptamer is a dipeptide, a polypeptide having 3 to 6 amino acids, a polypeptide of 7 amino acids, a polypeptide of 8 to 11 amino acids in length, a polypeptide of 12 amino acids, a polypeptide of 13 to 52 amino acids in length, or a polypeptide of greater than 52 amino acids in length. In another example embodiment, the linker has the same amino acid sequence as the peptide aptamer. In another example embodiment, the first condition is exposure to light, a decreased intensity of light or darkness, heat, pH, stress, ions, the isomer-specific affinity agent or combination thereof, and the second condition is exposure to light, a decreased intensity of light, darkness, heat, pH, stress, ions, the isomer-specific affinity agent or combination thereof that differs from the first condition. In another example embodiment, a) the first condition is exposure to broad wavelength of light on the visible spectrum or a portion thereof, and the second condition is exposure to a broad wavelength of light on the ultraviolet spectrum or a portion thereof, b) the first condition is exposure to wavelength of light on the visible spectrum or a portion thereof, and the second condition is exposure to a decreased intensity of light, or darkness, that is different from the first condition, or c) the first condition is exposure to a wavelength of light and the second condition is exposure to a wavelength of light differing from the first by a wavelength of more than 10 nanometers. In another example embodiment the isomerizable organic molecule is a photoisomerizable organic molecule. In yet another example embodiment, the photoisomerizable organic molecule has a spiropyran core structure or a derivatized form of spiropyran. When the isomerizable organic molecule is a photoisomerizable organic molecule, the first condition is a first wavelength of light and the second condition is a second wavelength of light.

In a second aspect, the invention is directed to methods for selectively binding and releasing a molecule of interest using the binding pair of the invention. According to an example embodiment, the method comprises: a) providing a solid substrate coated with an isomerizable organic molecule or covalently linked thereto (e.g., photoisomerizable organic molecule) in a first isomer configuration; b) contacting the coated solid substrate with a solution comprising a molecule of interest coupled to an isomer-specific affinity agent (e.g., affinity tag, affinity peptide, peptide aptamer, nucleic acid aptamer) under a first condition to form a complex between the affinity agent and the isomerizable organic molecule coated on the solid substrate; c) washing the complex formed in step (b) to remove matter not associated with the complex, such as non-specific binding; and d) converting the isomerizable organic molecule in the first isomer configuration to a second or subsequent isomer configuration under a second or subsequent condition (e.g., by applying a suitable isomerization-inducing agent), whereby the conversion of the molecule to the second or subsequent isomer configuration allows the molecule of interest to dissociate from the isomerizable organic molecule, thereby eluting the molecule of interest. In an example embodiment of the second aspect of the invention, the isomerizable organic molecule and the isomer-specific affinity agent together are the binding pair. In another example embodiment, the isomerizable organic molecule is a photoisomerizable organic molecule, such as an organic molecule having a spiropyran core structure or a derivatized form of spiropyran. In another embodiment, the suitable isomerization-inducing agent is a light source configured to apply a broad or specific wavelength of light, such as a light-emitting diode or a lamp. In another embodiment, the suitable isomerization-inducing agent is a heat source or a cooling source. In another embodiment, the method further comprises coating the solid substrate with the isomerizable organic molecule or covalently linking the isomerizable organic molecule to the solid substrate in the first isomer configuration prior to step (a).

In a third aspect, the invention pertains to a chromatographic apparatus (e.g., column, porous membrane or filter) for selectively binding and releasing a molecule of interest, comprising: a housing; a coated solid substrate (e.g., plurality of coated beads) packed within the housing, the coating being a photoisomerizable organic molecule in a first isomer configuration, a second or subsequent isomer configuration, or a combination of both; at least one light source of suitable wavelength to convert the photoisomerizable organic molecule in the first isomer configuration to a second or subsequent isomer configuration, wherein the coated solid substrate (e.g., plurality of coated beads) is in light contact with the at least one light source. In one embodiment of the third aspect of the invention, the chromatography apparatus is a column having a housing that is configured in a concentric cylinder where the center of the cylinder is hollow. The exterior of the housing and/or the hollow core of the cylinder comprise at least one light source that can be controllably illuminated. In another embodiment of the third aspect of the invention, only the exterior of the column comprises at least one light source that can be controllably illuminated.

In a fourth aspect, the invention pertains to kits for selectively binding a molecule of interest using the binding pairs described herein. A kit comprises a solid substrate coated with or covalently linked to a photoisomerizable organic molecule in a first isomer configuration, second or subsequent isomer configuration or combination thereof; an isomer-specific affinity agent having an affinity for the photoisomerizable organic molecule in the second or subsequent isomer configuration; and instructions for binding the affinity agent to a molecule of interest and subsequently selectively binding to and optionally releasing the affinity agent from the photoisomerizable organic molecule. In a version of the fourth aspect of the invention, the kit further comprises a light source of a broad or specific wavelength suitable to convert the photoisomerizable organic molecule in the first isomer configuration to a second or subsequent configuration.

The binding pairs of the invention and methods of selectively binding and releasing a molecule of interest using the binding pairs described herein can be used to purify, separate, isolate or pattern the molecule of interest. The binding of the molecule of interest to chromatographic media and its release is controlled by a change in the isomeric form of the isomerizable organic molecule, in the presence of light, temperature, pH, the isomer-specific affinity molecule or combinations of these. For example, light rather than solvent is used in the chromatographic apparatus (e.g., column) as the elution stimulus to elute the molecule of interest. The invention described herein allows for the rapid purification of a molecule of interest, such as a recombinant protein without requiring harsh or expensive elution buffers. Additionally, light is a convenient elution stimulus as it can be applied on both macro and nanometer scales, it does not fundamentally alter the chemical nature of the surrounding solvent, it can be administered in a very wide range of time scales, and it can be automated for sequential or simultaneous elution.

### BRIEF DESCRIPTION OF THE DRAWINGS

The foregoing will be apparent from the following more particular description of example embodiments of the invention, as illustrated in the accompanying drawings. The drawings are not necessarily to scale, emphasis instead being placed upon illustrating embodiments of the present invention.

FIGS. 1A-C depict differential binding affinities after photoisomerization. FIG. 1A shows a two-isomer state model where photoisomer A changes its chemical and/or structural properties to photoisomer B, in response to light. FIG. 1B shows another version of the two-component system using an isomer-specific aptamer. FIG. 1C shows a version of the two-component system where a surface or solid support is modified with an isomerizable organic molecule.

FIG. 2 is a bar graph representing different peptides binding to plates modified with a spiropyran molecule.

FIG. 3 is a line graph representing binding of peptides to beads selectively modified with a spiropyran molecule.

FIGS. 4A-C are illustrations of example purification columns before and after isomerization. FIG. 4A shows an example method for light-mediated binding and releasing of target proteins. FIG. 4B shows an example embodiment of a photoisomerizable organic molecule on a support (e.g., a cross-linked agarose bead or other chromatographic media), where the photoisomerizable organic molecule is bound to an affinity peptide or aptamer.

FIG. 4C shows an example embodiment of a chromatographic column that is adapted to have a housing that wraps around the column and at least one light source in contact with the column for controllably illuminating (e.g., sequentially or simultaneously illuminating) the derivatized beads with photoisomerizable organic molecule.

FIGS. 5A and 5B show the conversion of the merocyanin (MC) isoform by exposure to visible (Vis) light to the spiropyran (SP) isoform, with the disappearance of the dark hue indicating isomerization (red/orange hue—not shown).

FIGS. 6A-D show the isomerization of example photoisomerizable organic molecules.

FIG. 7 shows examples of bead conjugation techniques.

### DETAILED DESCRIPTION OF THE INVENTION

A two-component system is described for affinity separation of biological products, such as recombinant proteins, where the two-components can assemble and disassemble according to wavelength of light or other inducible condition, such as light, darkness or a decreased intensity of light, temperature, pH, the isomer-specific affinity agent or combination of these. The two-component system is comprised of an isomerizable organic molecule and an isomer-specific affinity agent. The invention takes advantage of isomerizable organic molecules (a first component in the two-component system) that have different isomeric forms, where in the first isomeric form the organic molecule will bind to a binding partner (a second component in the two-component system) and where in a second isomeric form of the organic molecule the binding partner will release or dissociate therefrom. The change between isomeric forms is induced by conditions such as light, a decreased intensity of light, darkness, temperature, pH, the isomer-specific affinity agent or combination of these, and as such the two-component system assembles and disassembles according to the inducing conditions. The two-component system uses an isomerizable organic molecule and its affinity binding partner to separate and/or purify recombinant proteins, for example, in a chromatographic column.

Using the two-component system of the invention, a molecule of interest can be bound and released using, for example, light as an elution stimulus. The invention describes a binding pair, comprising a first binding member being an isomerizable organic molecule, and a second binding member being an isomer-specific affinity agent. The isomerizable organic molecule has a binding affinity for the second binding member under a first condition, and a different binding affinity for the second binding member under the second condition. An example isomerizable organic molecule useful for the present invention is a photoisomerizable organic molecule. Because binding affinity can be controlled by the specific isomerization state of the photoisomerizable organic molecule, light can be used to control association between the isomer-specific affinity agent (or other molecular entities attached to the isomer-specific affinity agent) and the photoisomerizable organic molecule (or surfaces modified with the organic molecule). For example, the difference in binding affinity can be such that the first binding member and the second binding member associate under a first condition, and disassociate under a second condition. Light is useful in this regard as it can be applied on both macro and nanometer scales, does not fundamentally alter the chemical nature of the surrounding solvent, and can be administered in a very wide range of time scales.

Accordingly, an “isomerizable organic molecule,” as use herein, refers to an organic molecule which undergoes one or more structural or chemical changes in response to exposure (e.g., by a suitable isomerization-inducing agent) to a first condition or a second or subsequent condition. The first condition can be exposure to light, a decreased intensity of light, darkness, heat, pH, stress, ions, the isomer-specific affinity agent or a combination thereof. The isomerizable organic molecule can undergo one or more structural changes, including reversion to the previous structure or state, when exposed to a second or subsequent condition. The second or subsequent condition can be exposure to light, a decreased intensity of light, darkness, heat, pH, stress, ions, the isomer-specific affinity agent or combination thereof that differs from the first condition. Stress can be provided using a solvent to preferentially stabilize one isomeric from over another. Examples of suitable solvents include solvents such as, for example, water, water buffered with phosphate, dimethyl sulfoxide, tetrahydrofuran, dimethylformamide, ethanol, propanol, methanol and the like. For example, when a spiropyran molecule is placed into tetrahydrofuran, the molecule favors the open-ring (or merocyanin) configuration. Alternatively, for example, when a spiropyran molecule is placed into deionized water, the molecule favors the closed-ring configuration. Stress can also be provided using an ionic solution. Ions can be provided via an aqueous solvent with high ionic strength. An isomerizable organic molecule can take one configuration when exposed to a solvent with a high ion concentration, and a different configuration when exposed to a solvent with little or no ion concentration. An aqueous solvent with a dissolved ionic component or a high ionic strength can, for example, stabilize an open-ring configuration of a spiropyran molecule. Additionally, stress can be provided using an ionic solution to preferentially stabilize one isomer over the other isomer, such as by a divalent ionic solution. pH can be altered via exposure to a buffer. Examples of suitable buffers include acidic buffers with a pH between about 2 and about 3. Changes in temperature can also cause the isomerizable organic molecule to undergo one or more structural or chemical changes. For example, an isomerizable organic molecule may take one configuration at a temperature above 70° C. In another example, an isomerizable organic molecule may take a different configuration below 4° C. A heating source adds heat to promote change of the configuration of an isomerizable organic molecule. A cooling source cools or otherwise reduces the temperature to promote change of the configuration of an isomerizable organic molecule.

The term “alkyl,” as used herein, refers to both a saturated aliphatic branched or straight-chain monovalent hydrocarbon radical having the specified number of carbon atoms. Thus, “(C₁-C₆) alkyl” means a radical having from 1-6 carbon atoms in a linear or branched arrangement. Examples of “(C₁-C₆) alkyl” include, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl. Alkyl can be optionally substituted with halogen, —OH, oxo, (C₁-C₄) alkyl, (C₂-C₆) alkynyl, (C₁-C₆) alkoxy, (C₁-C₆) alkoxy(C₁-C₄)alkyl, nitro, cyano, azido, and —N(R^{a})(R^{b}) wherein R^{a} and R^{b} are each independently selected from —H and (C₁-C₃) alkyl.

The term “alkenyl,” as used herein, refers to a straight-chain or branched alkyl group having one or more carbon-carbon double bonds. Thus, “(C₂-C₆) alkenyl” means a radical having 2-6 carbon atoms in a linear or branched arrangement having one or more double bonds. Examples of alkenyl groups include, but are not limited to, ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene).

The term “alkynyl,” as used herein, refers to a straight-chain or branched alkyl group having one or more carbon-carbon triple bonds. Thus, “(C₂-C₆) alkynyl” means a radical having 2-6 carbon atoms in a linear or branched arrangement having one or more triple bonds. Examples of alkynyl groups include, but are not limited to, ethynyl, propynyl, butynyl, pentynyl, and the like. The one or more carbon-carbon triple bonds can be internal (such as in 2-butyne) or terminal (such as in 1-butyne).

The term “alkoxy”, as used herein, refers to an “alkyl-O—” group, wherein alkyl is defined above. Examples of alkoxy group include methoxy or ethoxy groups.

The term “amino,” as used herein, means an “—NH₂,” an “NHRₚ,” or an “NRₚR_{q},” group, wherein Rₚ and R_{q} may be any of the alkyl, alkenyl, alkynyl, alkoxy, cycloalkyl, cycloalkoxy, aryl, heteroaryl, and bicyclic carbocyclic groups. In the present invention, the amino may be primary (NH₂), secondary (NHRₚ) or tertiary (NRₚR_{q}). A (di)alkylamino group is an example of an amino group substituted with one or two alkyls.

The term “aryl,” as used herein, refers to an aromatic monocyclic or polycyclic (e.g. bicyclic or tricyclic) carbocyclic ring system. Thus, “(C₆-C₁₀) aryl” is a 6-10 membered monocylic or polycyclic system. Aryl systems include optionally substituted groups such as phenyl, biphenyl, naphthyl, phenanthryl, anthracenyl, pyrenyl, fluoranthyl or fluorenyl. An aryl can be optionally substituted. Examples of suitable substituents on an aryl include halogen, hydroxyl, (C₁-C₁₂) alkyl, (C₂-C₆) alkenyl, (C₂-C₆) alkynyl, (C₁-C₆) haloalkyl, (C₁-C₃) alkylamino, (C₁-C₃) dialkylamino (C₁-C₆) alkoxy, (C₆-C₁₀)arylamino, (C₆-C₁₀) aryl, (5-12 atom) heteroaryl, —NO₂, —CN, and oxo.

In some embodiments, a (C₆-C₁₀) aryl selected from phenyl, indenyl, or naphthyl. In some embodiments, a (C₆-C₁₀) aryl selected from phenyl or naphthalene.

The term “hetero,” as used herein refers to the replacement of at least one carbon atom member in a ring system with at least one heteroatom selected from N, S, and O. “Hetero” also refers to the replacement of at least one carbon atom member in a acyclic system. A hetero ring system or a hetero acyclic system may have 1, 2, or 3 carbon atom members replaced by a heteroatom.

The term “heteroaryl,” as used herein, refers aromatic groups containing one or more atoms is a heteroatom (O, S, or N). A heteroaryl group can be monocyclic or polycyclic, e.g., a monocyclic heteroaryl ring fused to one or more carbocyclic aromatic groups or other monocyclic heteroaryl groups. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl and azaindolyl.

The terms “halogen” or “halo,” as used herein, refer to fluorine, chlorine, bromine or iodine.

The term “oxo,” as used herein, refers to ═O. When an oxo group is a substituent on a carbon atom, they form a carbonyl group (C(O)).

A “photoisomerizable organic molecule,” as used herein, refers to a molecule which undergoes one or more structural or chemical changes in response to light. An example of a photosiomerizable organic molecule is a spiropyran having at least a first isomer configuration and a second or subsequent isomer configuration. “Spiropyran,” as used herein, refers to an optionally substituted 2H-pyran, wherein the hydrogen atom at position two has been replaced by a second ring system in a spiro manner as illustrated in the spiropyran core structure of formula (I) below:

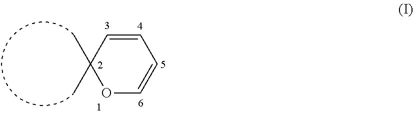

wherein the second ring system can be carbocyclic or heterocyclic. An example of a spiropyran having at least a first isomer configuration and a second or subsequent isomer configuration is a compound of formula (II) below:

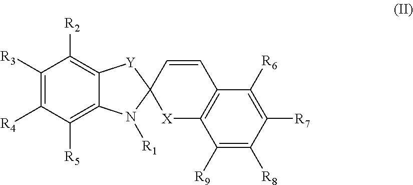

wherein R₁, R₂, R₃, R₄, R₅, R₆, R₇, R₈, and R₉ are the same or different and are each independently selected from hydrogen, (C₁-C₂₀) alkyl or substituted alkyl, (C₂-C₂₀) alkenyl, (C₂-C₂₀) alkynyl, (C₆-C₁₀) aryl, (5-12 atom) heteroaryl, (C₁-C₂₀) alkoxy, amino, halogen, cyano, trichloromethyl, trifluoromethyl, nitro, sulfur, maleimido —C(O)—OH, azido, or HO—(O)C—(C₁-C₃)alkyl;
X is oxygen; and
Y is Se or C(CH₃)₂.

In another example, the compound of formula (II) can be a polymer of formula (IIa):

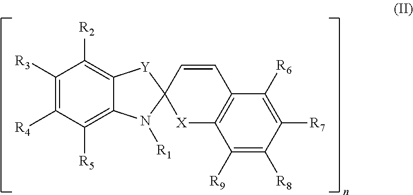

wherein R₁-R₉, X, and Y are defined above with respect to formula (II), and n is an integer greater than 1. In another example embodiment, n is between 1 and 500. The compound of formula (II) can polymerize at any point of attachment R₁-R₉. Additionally, the compound of formula (II) can bind to a solid substrate at any point of attachment R₁-R₉.

Examples of photoisomerizable organic molecules of the present invention include compounds of formula (III), (IV-1), (IV-2), (V-1) and (V-2):

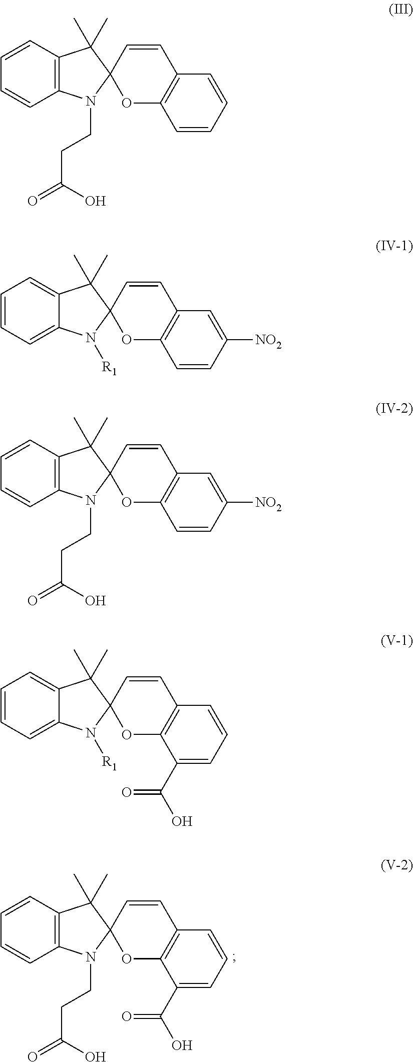

wherein R₁ is defined above with respect to formula (II).

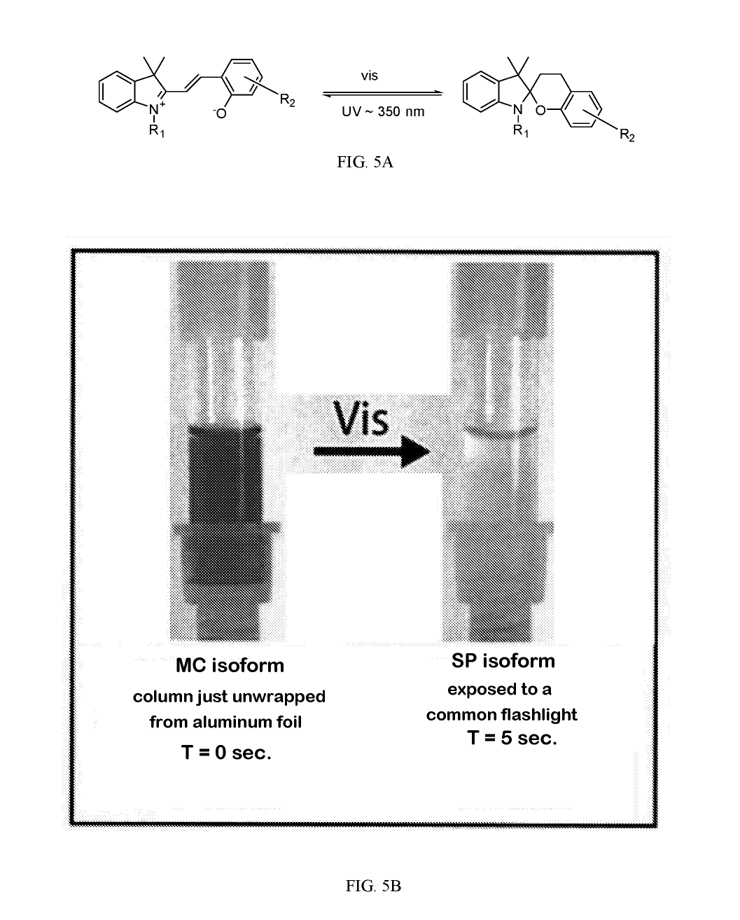

An example of the first isomer configuration is a compound of formula (Ma), which when exposed to a first condition (such as light in the visible spectrum) converts to a compound of formula (III). An example of the second isomer configuration is a compound of formula (III), which when exposed to a second condition (such as ultraviolet light) converts to a compound of formula (Ma). The isomerization represented below is merely to illustrate the concept and is not intended to be limiting to the reaction or structures set out below, and is similarly represented in FIG. 5A.

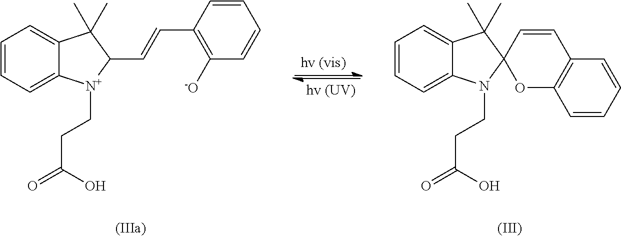

It is understood that the isomers described herein exist in equilibrium, that is, using the example compound of formula (III), for a given sample, there is a concentration of both compounds of formula (III) and compounds of formula (Ma). This also holds true for compounds of formula (IV-1) and (IV-1a), (IV-2) and (IV-2a), (V-1) and (V-1a), and (V-2) and (V-2a), wherein (IV-1a), (IV-2a), (V-1a) and (V-2a) are as follows:

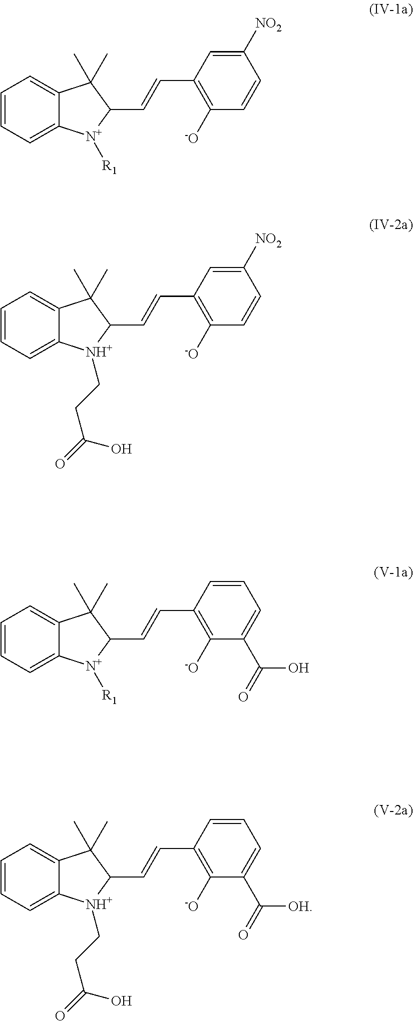

Accordingly, “isomerization” refers to altering the concentration of the isomers in a given sample such that the concentration of one isomer decreases and another isomer increases as the molecules undergo chemical or structural isomerization, or otherwise exposing the isomers to a medium which favors one isomer over another, thereby increasing the concentration of one isomer in favor of another.

When referring to a “photoisomerizable organic molecule,” it is understood that reference is also made to an “isomerizable organic molecule” in general.

Additional examples of photoisomerizable organic molecules suitable for use in the present invention include, but are not limited to, diaryethenes, specifically stilbenes and dithienylethenes; azobenzenes; spiropyrans; fulgides; naphthacenequinones; hindered alkanes, crown ethers; retinoids; cyanine dyes; and polymers of these. An example of a hindered alkane is a compound of formula (VI):

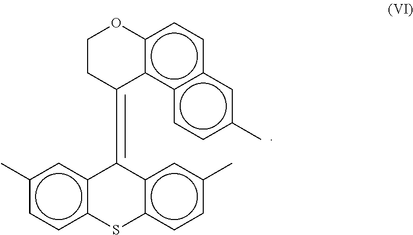

Photoisomerizable organic molecules can be conjugated, bound, or otherwise coated onto the surface of a solid substrate via covalent or non-covalent means, providing a light-controlled surface characteristic. This characteristic allows the solid surface to interact with isomer-specific affinity agent, regardless of hydrophobicity, charge change or development, conformational change or a combination of these qualities. Affinity peptides typically require high affinity to purify fusion proteins from crude lysate, and therefore an example photoisomerizable organic molecule (otherwise known as a photoisomerizable affinity molecule, or “PIAM”) has a large free energy difference between the isomeric states, typically cis and trans (e.g., preferably greater than 20 kJ/mole). Free energy of peptide binding tends to stabilize the initial, peptide-bound state. Thus, a large, light-catalyzed “driving force” allows peptide tagged proteins to quickly disassociate from the matrix.

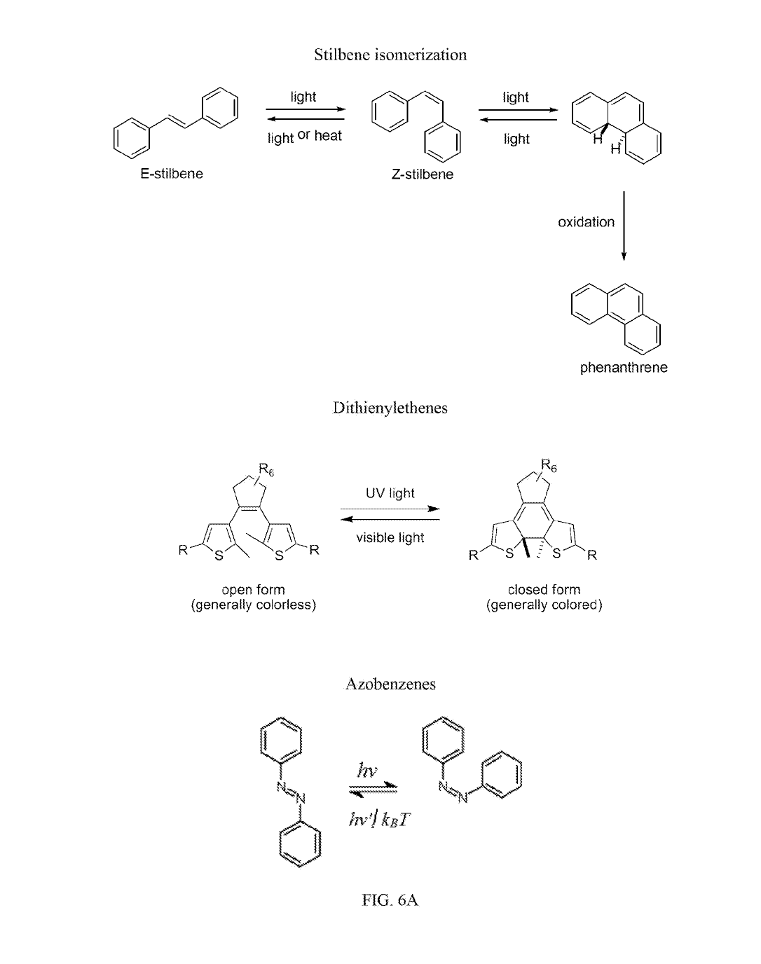

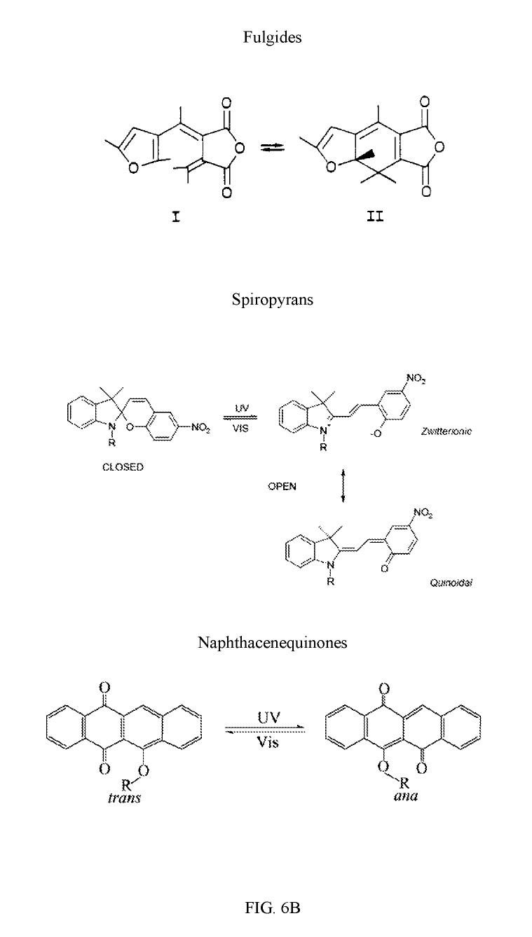

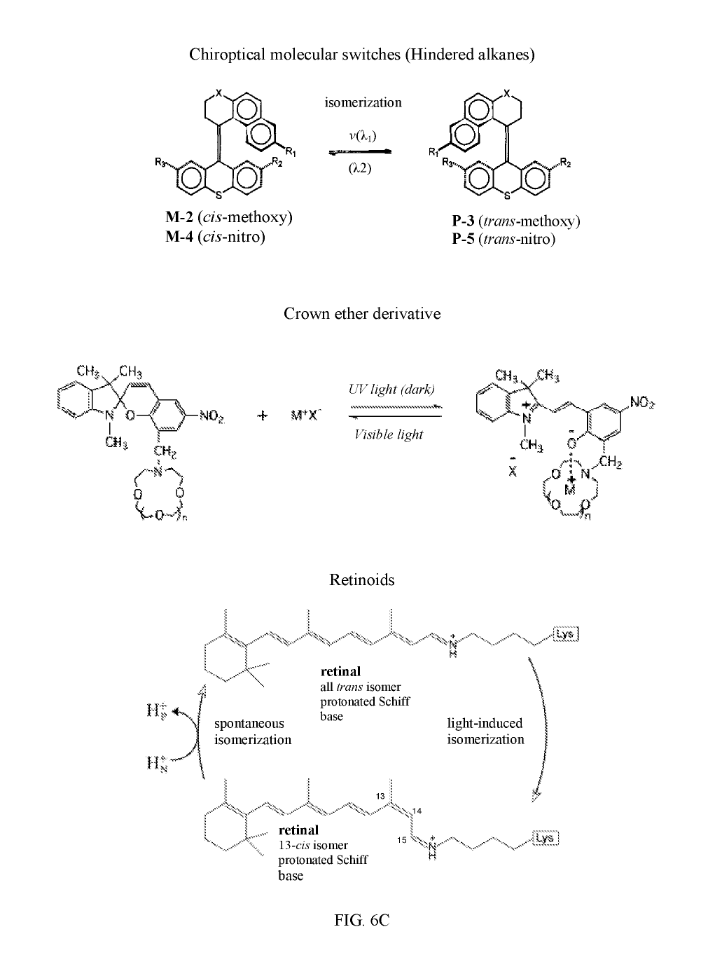

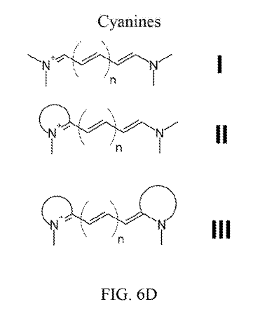

Other suitable characteristics of PIAM molecules are: 1) ability to isomerize in water and/or at physiological pH; 2) substantially no reaction with common buffers, such as Tris, HEPES, phosphate; 3) a charge characteristic, preferably a positive charge; 4) substantially no degradation in ethanol; 5) isomerization in reducing conditions as well as non-reducing conditions; 6) isomerization in various salt concentrations (e.g., 0-400 mM NaCl); and 7) capable of forming a stable and high-efficiency conjugation to agarose matrix. Given the listed constraints, the following compounds represent examples of suitable classes of PIAM molecules: 1) diaryethenes, specifically stilbenes and dithienylethenes; 2) azobenzenes; 3) fulgides; 4) spiropyrans; 5) naphthacenequinones; 6) hindered alkanes; 7) crown ether derivatives; 8) retinoids; and 9) cyanine dyes. Additional example compounds include combinations of the above, such as polymeric azobenzenes, stilbenes or spiropyrans. See FIGS. 6A-D.

A “light source,” as used herein, refers to any light generating element which is capable of generating light at a specific wavelength or range of wavelengths, the specific wavelength or wavelengths being suitable to convert the isomerization state of a photosiomerizable organic molecule, as defined herein. For example, in an embodiment of the invention, when the photoisomerizable organic molecule is a molecule of formula (III), illustrated above, application of a specific wavelength of ultraviolet light converts the first isomer to the second isomer, illustrated by formula (Ma). Examples of suitable wavelength range includes, but are not limited to, the visible (vis) spectrum or a portion thereof, the ultraviolet (UV) spectrum or a portion thereof, and the infrared spectrum (IR) or a portion thereof. An example wavelength suitable to convert the first isomer to the second isomer is 350 nm. In another embodiment, exposure to light is sufficient to induce a change in the isomerization state of a photoisomerizable organic molecule. For example, a photoisomerizable organic molecule can exist in a first configuration when exposed to minimal or no light (e.g., when placed in the dark), and exist in a second configuration when exposed to light from any light-emitting device, including, for example, a lamp. A lamp can produce light via an incandescent bulb, a halogen bulb, or via gas-discharge, such as, for example, use of a gas (e.g., argon, neon, krypton, or xenon, or a combination of said gases).

An “isomer-specific affinity agent” or an “affinity tag,” as used herein, refers to a peptide sequence or an oligonucleotide sequence bound to a target molecule of interest with an affinity for a particular protein, antibody, drug, nucleic acid, polymer, organic molecule, metal ion, peptide, or enzyme and which exhibits differential affinity for a single or subset of multiple isomerization states of the isomerizable organic molecule. Accordingly, the affinity tag can be used to facilitate separation of the target molecule of interest from a crude biological source using an affinity technique. The affinity tag can be bound covalently, such as through a chemical bond, or non-covalently, such as through hydrogen-bonding or electrostatic interactions. An affinity technique relies on a specific interaction between the affinity tag and another structure, such as but not limited to a protein, an antibody, an enzyme, a drug, a substrate, a receptor or a ligand. An example affinity tag includes an aptamer. An “aptamer,” as used herein, refers to a molecule that facilitates high-affinity binding to a specific molecule of interest. Example aptamers can be peptides, oligonucleic acids, or nucleic acids. A “peptide aptamer,” as used herein, refers to a natural or synthetic peptide molecule that binds to a specific target molecule. A “nucleic acid aptamer,” as used herein, refers to a natural or synthetic nucleic acid sequence such as, for example, an RNA aptamer, a DNA aptamer, or a reduced microRNA aptamer.

In some embodiments, the peptide aptamer is a di peptide. In some embodiments, the peptide aptamer is a polypeptide comprising 3 to 6 amino acids in length. In some embodiments, the peptide aptamer is a polypeptide comprising 3, 4, 5, or 6 amino acids in length. In some embodiments, the peptide aptamer is a polypeptide comprising 7 amino acids in length. In another embodiment, the peptide aptamer is a polypeptide comprising 8 to 11 amino acids in length. In some embodiments, the peptide aptamer is a polypeptide comprising 8, 9, 10, or 11 amino acids in length. In some embodiments, the peptide aptamer is a polypeptide comprising 12 amino acids in length. In some embodiments, the peptide aptamer is a polypeptide comprising 13 to 52 amino acids in length. In some embodiments, the peptide aptamer is a polypeptide comprising 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 amino acids in length. In another embodiment, the peptide aptamer is a polypeptide comprising greater than 52 amino acids. In another embodiment, the peptide apatmer comprises less than 120 amino acids. In another embodiment, the peptide aptamer is a polypeptide comprising 3 to 18 amino acids in length. In another embodiment, the peptide aptamer is a polypeptide comprising 3, 4, 5, 6, 7, 8, 9, 10, 11, 12, 13, 14, 15, 16, 17, or 18 amino acids in length. “Amino acid” as used herein refers to either a natural or modified amino acid. An example of a seven unit polypeptide is: LAREPTS (SEQ ID NO: 1). An example of a twelve unit polypeptide is: TLRVPPNPNMNV (SEQ ID NO: 53). In another example embodiment, the peptide aptamer is the peptide sequence EYSPKLFPPHRL (SEQ ID NO: 56). The following peptide sequences represent example peptide aptamers suitable for use in the present invention, but are not limited to, the peptide sequences shown in Table 1. The sequences shown in Table 1 may have a glycine appended to the C-terminus or may have one or more amino acids appended to either the N-terminus or the C-terminus. It should be understood that a suitable aptamer can be designed to bind a set of isomers of similar structure and is well within the skill of the artisan to make and design such aptamers. It should also be understood that a person of skill in the art would be able to create an aptamer of any length, provided that it is long enough to interact with the isomerizable organic molecule. Accordingly, a person having skill in the art would be able to design an aptamer of any length, and the aptamer would fall within the confines of the present invention.

TABLE 1

| | SEQ ID |
|---|---|
| Aptamer | NO: |
| LAREPTS | 1 |
| HELVRSP | 2 |
| WALDRGA | 3 |
| APSTPTP | 4 |
| SMPQTAG | 5 |
| EPLQLKM | 6 |
| ATPLWLK | 7 |
| AKIDART | 8 |
| YNHQRPP | 9 |
| ALIPKPR | 10 |
| QLITKPL | 11 |
| STPIQQP | 12 |
| TFAKSAY | 13 |
| STFTKSP | 14 |
| TPAHNDY | 15 |
| SLSLIQT | 16 |
| NQDVPLF | 17 |
| GPHLGLK | 18 |
| LAGPQMH | 19 |
| SQASSLK | 20 |
| SDLSSPY | 21 |
| LVTTWPA | 22 |
| ALYKNTS | 23 |
| QQISTQM | 24 |
| AWLPAGA | 25 |
| GTWLSRG | 26 |
| GHQVSRL | 27 |
| GPMLARG | 28 |
| TASLVRP | 29 |
| TDSLRLL | 30 |
| AKPDWRF | 31 |
| VQTYARV | 32 |
| YTEPVRE | 33 |
| MSLQQEH | 34 |
| MKWQTV | 35 |
| NERALTL | 36 |
| SPSTHWK | 37 |
| WSTTNVP | 38 |
| YHKLDPL | 39 |
| LSNNNLR | 40 |
| LPAGRVL | 41 |
| STSFWIT | 42 |
| LGSPMSN | 43 |
| WELRPRT | 44 |
| TNIESLK | 45 |
| LYAEVIR | 46 |
| NHSTQMY | 47 |
| NLQIYAV | 48 |
| YDTSSAS | 49 |
| MHSNTWD | 50 |
| TPTTVSY | 51 |
| TSHSILQ | 52 |
| TLRVPPNPNMNV | 53 |
| HVKLVAVADLMN | 54 |
| YHPNGMNPYTKA | 55 |
| EYSPKLFPPHRL | 56 |
| HHLTHANSLTNT | 57 |
| WHWGLLYPASAN | 58 |
| KPLMTYKVIHYV | 59 |
| QPDLSHPSTNAY | 60 |
| FPTNLATRSAMV | 61 |
| LAGPQMHGK | 62 |
| PLRPKSEYPFHY | 63 |
| SYSPKLFPPHRL | 64 |
| SDLSSPYG | 65 |
| SDLSSPYGG | 66 |
| SDLSSPYGGC | 67 |
| EYSPKLFPPHRLGK | 68 |

The isomer-specific affinity agent can further comprise a linker bound to the peptide aptamer or the nucleic acid aptamer. A “linker,” as used herein, refers to a natural or synthetic polypeptide or nucleic acid sequence that is bound to the aptamer and is capable of binding to a molecule of interest. A linker can provide a sufficient amount of extra length to the isomer-specific affinity agent such that any potential steric limitations are overcome when the isomer-specific affinity agent interacts with the isomerizable organic molecule. The linker, ultimately, can provide rotational freedom which allows the isomer-specific affinity agent to associate with the isomerizable organic molecule. With respect to the linker, the term “bound” refers to a covalent bond between the linker, the aptamer, and the molecule of interest (e.g., the linker is covalently bound to the aptamer and the molecule of interest). The linker can be the same amino acid or nucleic acid sequence as the peptide or nucleic acid aptamer or it can be different. A linker can be simultaneously bound to both the aptamer and the molecule of interest, as illustrated by formula (V) below:

[aptamer]-[linker]-[molecule of interest] (V).

Phage display can be used for determining high-affinity peptide sequences which bind a particular isoform of the PIAM. For a given PIAM/matrix combination, conjugated PIAMs can be homogenized into either cis or trans isomers using the appropriate wavelength of light. Phage peptide library can be bound and the matrix washed with high-salt buffer to remove non-specific binders. Specific phage binders can then be eluted after switching the wave length of light to promote isomerization of the PIAM molecules. This process can be repeated under high stringency conditions to find micromolar and nanomolar affinity phage peptides that specifically elute with photoisomerization. Successful heptapeptides can be tested as affinity tags for recombinantly expressed proteins.

“Washing,” as used herein, refers to removal of substantially all matter which is not bound or associated to the photoisomerizable affinity molecule. It should be understood that in the washing process, not all matter may be removed. A trace amount of residual matter may be acceptable.

A “molecule of interest,” as used herein, is any molecule or structure that the user desires. For example, a molecule of interest can be a protein, an antibody, a drug, nucleic acid, a polymer, an organic molecule, a metal ion, a peptide or an enzyme of interest. The methods and compositions of the invention can be used to purify, isolate, selectively bind, selectively release, separate or pattern the molecule of interest. A molecule of interest can be produced via genetic engineering to include an isomer-specific affinity agent or affinity tag such as the ones described above. A molecule of interest can be produced via any method known to those of skill in the art, such as via use of a plasmid vector designed to promote synthesis of proteins modified with an affinity tag.

“Associated,” as used herein, refers to the capture, binding or assembly of the binding pair such as, for example, the photoisomerizable organic molecule and the isomer-specific affinity agent or affinity tag. Similarly, “disassociated,” as used herein, refers to the release or disassembly of the binding pair, such as, for example, the photoisomerizable organic molecule and the isomer-specific affinity agent or affinity tag. “Affinity,” as used herein, refers to the strength of the binding interaction between two binding partners of a binding pair such as, for example, the photoisomerizable organic molecule and the affinity tag. The association between the binding pair can be very highly specific such that an example affinity tag may only bind to a photoisomer when it is atomically flat. The suggestion that an affinity tag only binds to an atomically flat isomer is merely to provide an illustration of the concept of the present invention and is not intended to be limited to any one particular theory or atomic configuration.

A “solid substrate” as used herein can include a porous or non-porous material that can have any one of a number of shapes and forms, such as a strip, rod, particle, bead (solid or gel), mesh, membrane and gel (including hydrogel). Suitable materials are well known in the art and can include, for example, dextran, agarose, resin, paper, filter paper, nylon, glass, ceramic, silica, alumina, diatomaceous earth, cellulose, polymethacrylate, polypropylene, polystyrene, polyethylene, polyvinylchloride, and derivatives of each thereof. The solid substrate can be the sides and bottom of a column or can be a material added to a column. Examples of a solid substrate include agarose beads, immunoassay plates, cell culture plates, and the like. The solid substrate can be used to immobilize the molecule of interest, as defined above.

The solid substrate can be coated by forming a covalent or non-covalent bond with, for example, the isomerizable organic molecule. In an example embodiment, the isomerizable organic molecule can coat the solid substrate by means of a covalent bond. The covalent bond can be formed by any method known to a person of skill in the art, including, for example, click chemistry and peptide bond formation, EDC/NHS coupling via COOH or amine. In another aspect of the invention, the isomerizable organic molecule can be covalently attached to the solid substrate via a single-point attachment or via multi-point attachment, using methods known in the art. The point of attachment(s) should be selected such that the isomerizable effect is not hindered.

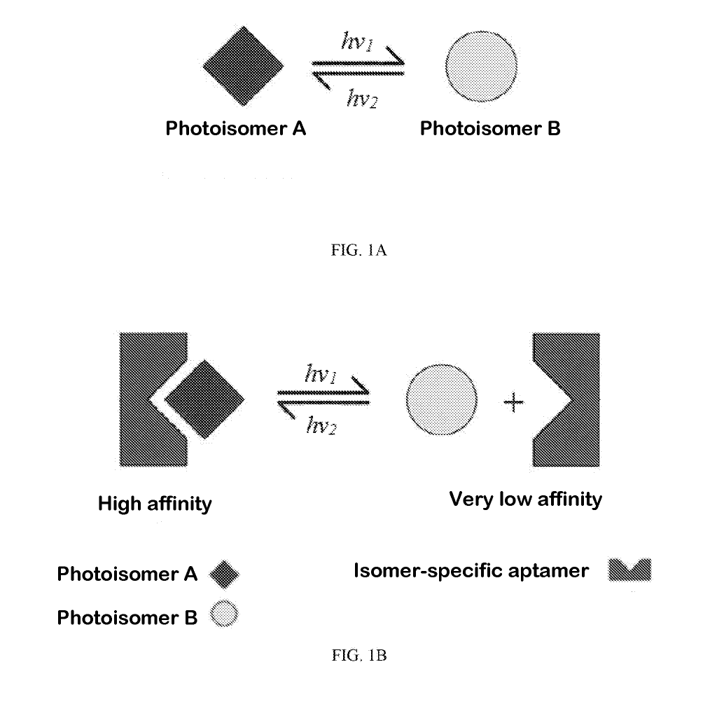

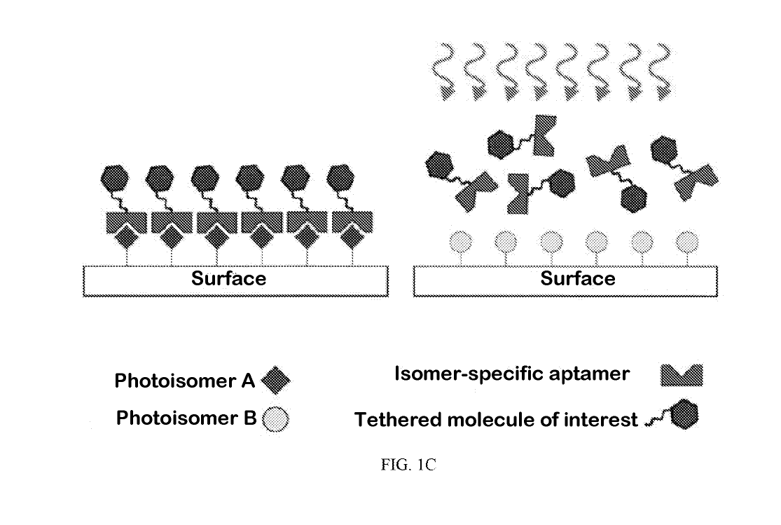

Example embodiments will now be described and illustrated in the figures. FIG. 1A is a schematic depicting differential binding affinities after photoisomerization. For example, certain molecules change their chemical and/or structural properties in response to light. Referring to FIG. 1A, hv₁ and hv₂ refer to wavelengths of light chosen to manipulate the isomerization state of the molecule. In the example illustrated in FIG. 1A, the molecule is a two-state model, but isomerizable organic molecules can have multiple chemical and structural isomers.

Referring to FIG. 1B, an example embodiment of the present invention can be composed of two components, an isomerizable organic molecule, such as a photoisomerizable organic molecule, which has a high binding affinity for one or more chemical isomers of the photoisomerizable organic molecule which, in turn, enables binding to be controlled with light. Referring to the example in FIG. 1B, Photoisomer A binds with high affinity to the isomer-specific aptamer, whereas Photoisomer B has very low affinity for the isomer-specific aptamer.

FIG. 1C is an illustration of a surface modified with an example isomerizable organic molecule. In FIG. 1C, the isomer-specific aptamer is bound a tethered molecule of interest, also referred to as an analyte of interest. The isomer-specific aptamer binds to Photoisomer A on the modified surface. When exposed to isomerizing light, the photoisomerizable organic molecule undergoes a structural change to Photoisomer B, thus releasing the isomer-specific aptamer and the molecule of interest from the modified surface.

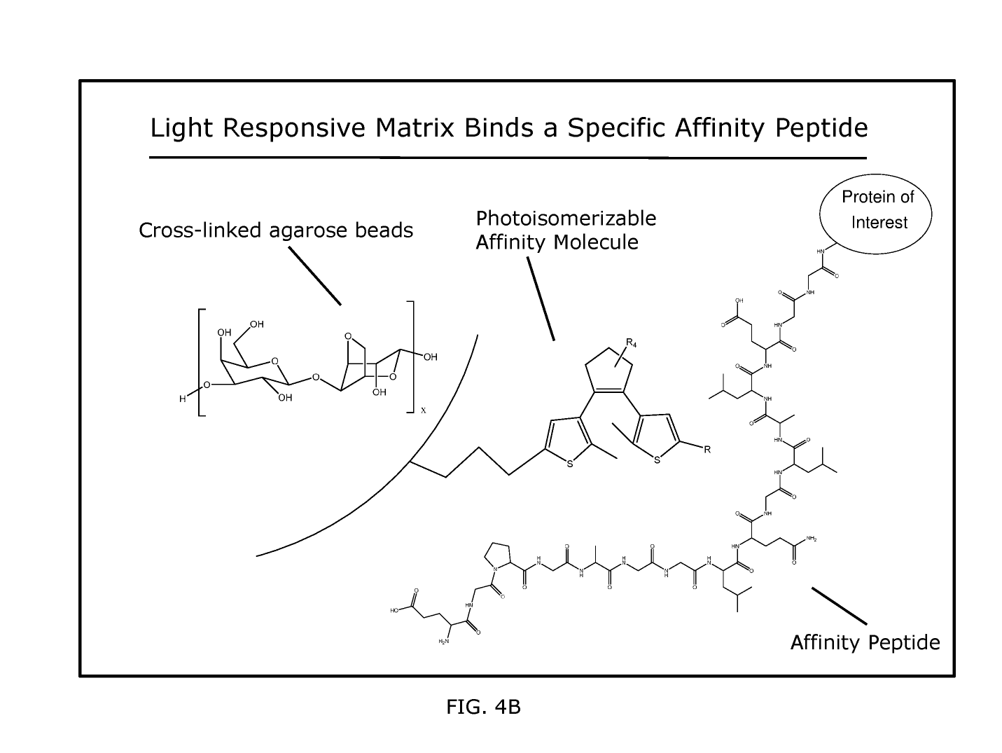

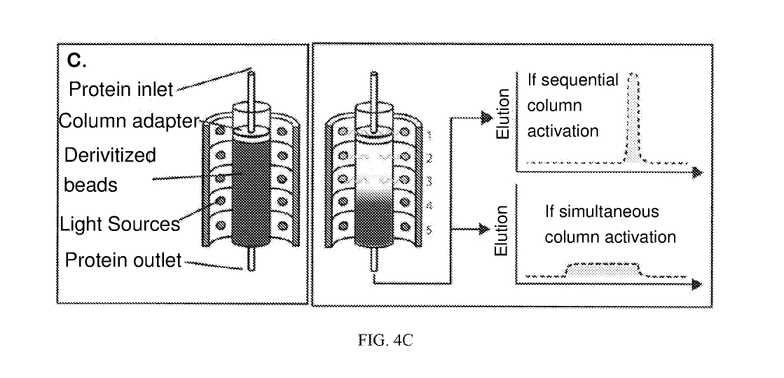

Photoisomerizable organic agents may have many uses. In an example embodiment, a light-responsive binding between a photoisomerizable small organic molecule and an affinity tag can be used to purify recombinant proteins from solution. In an example embodiment, the present invention is a method comprising providing a solid substrate, such as a bead modified, coated with, or covalently linked to a photoisomerizable organic molecule (e.g., spiropyran). See FIG. 4A. The solid substrate is contacted with a solution comprising a molecule of interest coupled to an affinity tag (e.g., peptide B7) under conditions to form a complex between the affinity tag and the isomerizable organic molecule coated on the solid substrate. An illustration of this complex is shown in FIG. 4B. Associating the affinity tag with the coated solid substrate can also be referred to as “pull-down” via a pull-down assay. The solid substrate is washed to remove matter not associated with the complex. The photoisomerizable organic molecule is then exposed to a suitable isomerization-inducing agent (e.g., light) to change the isomerizable organic molecule's structure into a different isomeric form which no longer binds to the affinity tag, thus eluting the molecule of interest. The above method can be carried out in a column, for example as illustrated in FIG. 4A and FIG. 4C.

In another embodiment, the invention is a column housing with sequential or simultaneous deployment of lights to create a more concentrated elution peak. FIG. 4C illustrates a chromatographic column for selectively binding and releasing the molecule of interest, the column comprising a housing, a plurality of coated beads packed within the housing, the coating being a photoisomerizable organic molecule in a first isomer configuration, and at least one light source of suitable wavelength to convert the photoisomerizable organic molecule in the first isomer configuration to a second isomer configuration, wherein the plurality of coated beads are in light contact with the at least one light source. As illustrated in FIG. 4C, the column can have multiple light sources surrounding the housing packed with the coated beads. The light sources can be activated sequentially or simultaneously. Referring to FIG. 4C, the light sources are labeled 1-5. Light source 1 can activate first, followed by 2, followed by 3, followed by 4, and then 5. Sequential activation of the light sources 1-5 can create a sharp elution peak, as illustrated in FIG. 4C. Alternatively, if all light sources 1-5 are activated simultaneously, the elution peak will be flat.

FIG. 5B illustrates the color/hue of the column of FIG. 4C at T=0 sec, when the column has been shielded from visible light, and again at T=5 sec, when the column has been exposed to visible light. The disappearance of the dark hue indicates isomerization of the isomerizable organic molecule. Red/orange hue not shown.

### Examples

### Synthesis of photoisomerizable affinity molecule (1-(β-carboxyethyl)-3′,3′-dimethylspiro(2′H-1′-benzopyran-2,2′-indoline)) (Formula (II))

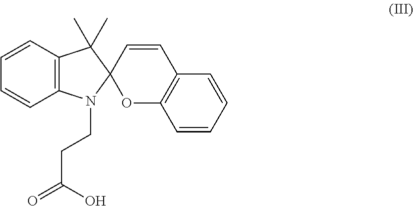 [SMILES: CC1(C)c2ccccc2N(CCC(=O)O)C12C=Cc1ccccc1O2]

A mixture solution of 2,3,3-trimethylindolenine (6 mL, 37 mmol) and 3-iodopropanoic acid (7.47 g, 37 mmol) was refluxed in 2-butanone (7 mL) under argon (or nitrogen) at 100° C. for 3 h. After evaporation of the solvent using a rotavap, the resulting solid material was dissolved in water and washed with dichloromethane. The aqueous phase was recovered and water was evaporated using the rotavap. The Fisher's base 1-(β-carboxyethyl)-2,3,3-trimethylindolenium iodide (white powder) noted CE-TMI was obtained without further purification in a typical yield of 73% (9.6 g). The previous iodide salt (8.17 g, 23 mmol) was mixed with 5-nitrosalicyladhehyde (3.85, 23 mmol) and piperidine (2.4 mL, 23 mmol) and 2-butanone (10 mL), and the mixture was refluxed for 3 h at 100° C. (Methyl ethyl ketone may replace the butanone). Then, the mixture was left overnight at room temperature and the product recrystallized as a yellow powder. This was filtered and washed with methanol. SP—COOH was recovered as a yellow powder after drying under vacuum overnight (5.1 g, 58% yield). See FIG. 5A.

### Differential Binding

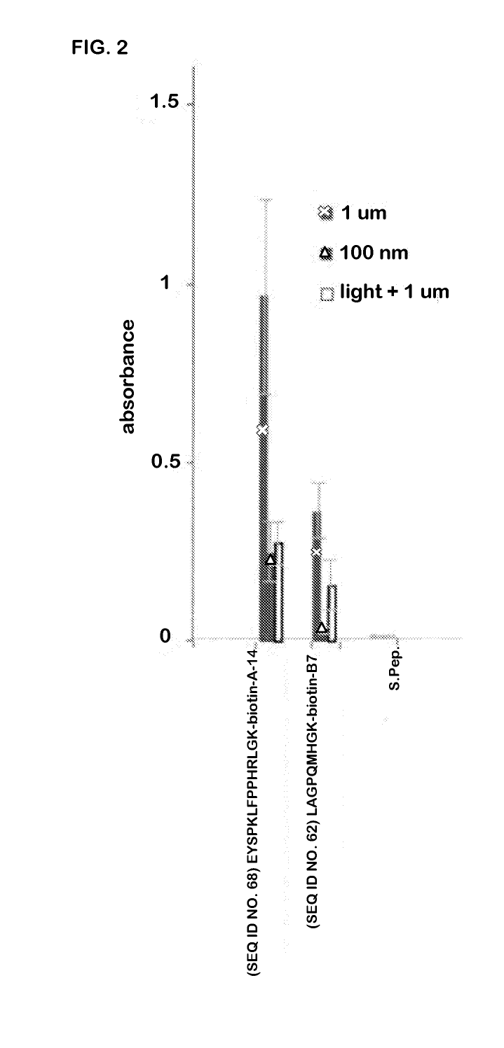

FIG. 2 is a bar graph representing different peptides binding to plates modified with a spiropyran molecule. Two biotinylated peptides were tested, EYSPKLFPPHRLGK (SEQ ID NO: 56) and LAGPQMHGK (SEQ ID NO: 62), for their affinity to bind to spiropyran modified plates, as well as S. pep which acted as a biotinylated negative control peptide. The absorbance reflects how much biotinylated peptide remained bound to the spiropyran modified plates after washing. The incubating and washing conditions are described below.

Biotinylated peptide was incubated at the indicated concentration (light gray: 1 μM; dark gray: 100 nm; white: 1 μM plus exposure to light), washed 10 times, then incubated with HRP-conjugated streptavidin. The washing was repeated 10 times, then plates were developed with 3,3′,5,5′-Tetramethylbenzidine (TMB) substrate and quantified in a 96 well plate reader. In all cases, biotin is covalently linked via the amine on the C-terminal lysine residue.

Peptides were incubated for 30 minutes at the indicated concentration. Plate was washed 10× in a Tecan Hydroflex 96 well plate washer with phosphate buffered saline. Plate was blocked with 1% BSA for 2 hours. Streptavidin conjugated to HRP was added for 1 hour. The plate was again washed 10× in a plate washer with PBS. TMB/H202 commercial ELISA substrate was added and H2SO4 was added to quench the reaction after 15 minutes of developing.

### Use of Spiropyran Modified Beads to Pull Down Biotin

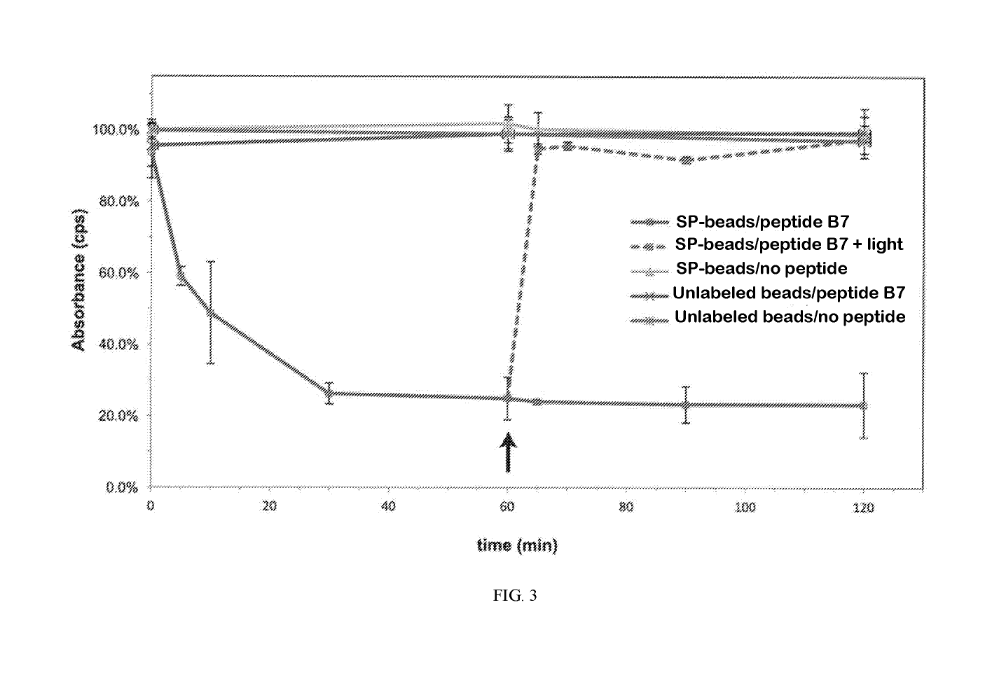

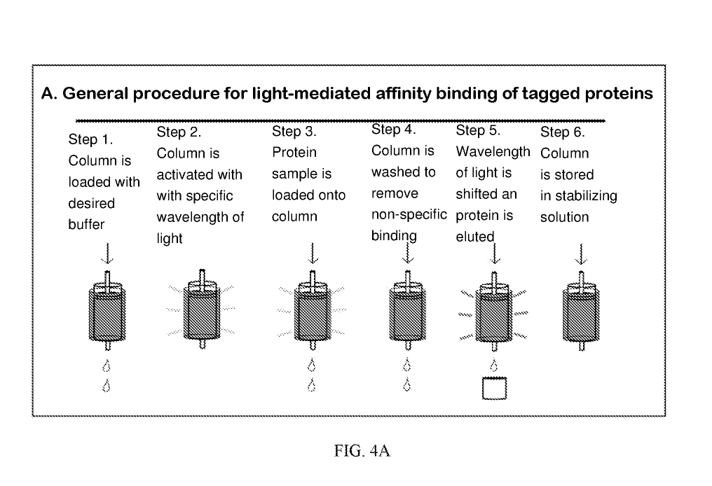

FIG. 3 is a line graph representing binding of peptides to beads selectively modified with a spiropyran molecule. The selectively modified beads were incubated with 10 μM of peptide B7, LAGPQMHGK (SEQ ID NO: 62), for the indicate length of time, and then washed twice with phosphate buffered saline (PBS). The beads were when incubated with 1 μM horseradish (HRP)-conjugated streptavidin, and the residual HRP-streptavidin was measured in solution. FIG. 3 illustrates the % absorbance of the beads after the indicated period of time. The higher the absorbance, the higher the concentration of streptavidin in the bulk solution. As indicated by the arrow at time=60 minutes, when the spiropyran modified beads were exposed to light at time=60 minutes, the streptavidin was released back into the bulk solution.

All experiments are N=3 (error bars are standard deviation) with the exception of “SP-beads/peptide B7,” which is n=6, then the samples were split in half with N=3 exposed to light (T=60 sec) and n=3 continued in darkness as indicated.

### Method for Agarose Bead Modification

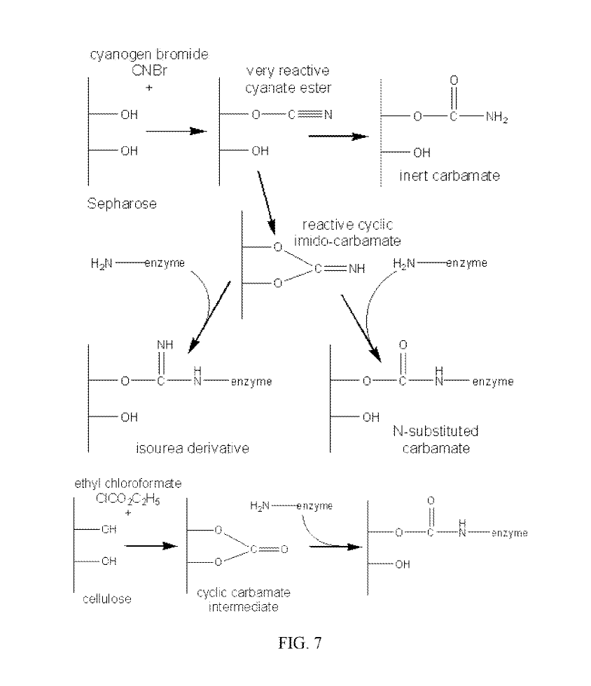

Wash 1 mL agarose beads with 2 column volumes DMSO (take from bottle in anhydrous fashion). In different container, mix 2.5 mg spiropyran and 80 mg EDC and 100 mg of NHS. Dissolve these compounds in DMSO; wait for 10 minutes at room temp. Add beads (which are now in DMSO) to this mixture. Make sure good mixing/suspension, i.e. enough DMSO to fully suspend the entire matrix. Agitate for 12 hours. Wash modified matrix with DMSO×20 c.v. to wash out free compounds. Remove DMSO with water was ×20 column volumes. Then store in water or 20% ethanol. See FIG. 7 for examples of bead conjugation techniques.

### REFERENCES

Liu, Y., Fan, M., Zhang, S., Sheng, X. & Yao, J. Basic amino acid induced isomerization of a spiropyran: towards visual recognition of basic amino acids in water. New J Chem (2007).
Shiraishi, Y., Adachi, K., Itoh, M. & Hirai, T. Spiropyran as a Selective, Sensitive, and Reproducible Cyanide Anion Receptor. Org. Lett. 11, 3482-3485 (2009).

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A binding pair, comprising:
a first binding member being an isomerizable organic molecule; and
a second binding member being an isomer-specific affinity agent,
wherein the isomerizable organic molecule has a binding affinity for the second binding member under a first condition, and a different binding affinity for the second binding member under a second condition.

2. The binding pair of claim 1, wherein the difference in binding affinity is such that the first binding member and the second binding member associate under a first condition, and disassociate under a second condition.

3. The binding pair of claim 1, wherein the isomer-specific affinity agent comprises a peptide aptamer or a nucleic acid aptamer.

4. The binding pair of claim 3, wherein the isomer-specific affinity agent further comprises a linker covalently linked to the peptide aptamer or the nucleic acid aptamer, wherein the linker is capable of non-covalently or covalently binding a molecule of interest.

5. The binding pair of claim 3, wherein the peptide aptamer is:
a) a dipepetide;
b) a polypeptide having 3 to 6 amino acids;
c) a polypeptide having 7 amino acids;
d) a polypeptide having 8 to 11 amino acids;
e) a polypeptide having 12 amino acids;
f) a polypeptide having 13 to 52 amino acids; or
g) a polypeptide having greater than 52 amino acids.

6. The binding pair of claim 3, wherein the peptide aptamer is a polypeptide having 3 to 18 amino acids.

7. The binding pair claim 1, wherein the linker comprises a protease sensitive cleavage site.

8. The binding pair of claim 1, wherein the first condition is exposure to light, light of decreased intensity, darkness, heat, pH, stress, ions, the isomer-specific affinity agent or combination thereof, and the second condition is exposure to light, light of decreased intensity, darkness, heat, pH, stress, ions, the isomer-specific affinity agent or combination thereof that differs from the first condition.

9. The binding pair of claim 1, wherein:
a) the first condition is exposure to broad wavelength of light on the visible spectrum or a portion thereof, and the second condition is exposure to a broad wavelength of light on the ultraviolet spectrum or portion thereof;
b) the first condition is exposure to wavelength of light on the visible spectrum or a portion thereof, and the second condition is exposure to a decreased intensity of light, or darkness, that is different from the first condition; or
c) the first condition is exposure to a wavelength of light and the second condition is exposure to a wavelength of light differing from the first by a wavelength of more than 10 nanometers.

10. The binding pair claim 1, wherein the isomerizable organic molecule is a photoisomerizable organic molecule.

11. The binding pair of claim 10, wherein the photoisomerizable organic molecule has a spiropyran core structure.

12. The binding pair of claim 11, wherein the photoisomerizable organic molecule has the spiropyran structure of formula (I): 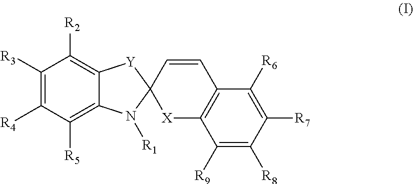
wherein R₁, R₂, R₃, R₄, R₅, R₆, R₇, R₈, and R₉ are the same or different and are each independently selected from hydrogen, (C₁-C₂₀) alkyl or substituted alkyl, (C₂-C₂₀) alkenyl, (C₂-C₂₀) alkynyl, (C₆-C₁₀) aryl, (5-12 atom) heteroaryl, (C₁-C₂₀) alkoxy, amino, halogen, cyano, trichloromethyl, trifluoromethyl, nitro, maleimido, sulfur, —C(O)—OH, azido or HO—(O)C—(C₁-C₃)alkyl;
X is oxygen and
Y is Se or C(CH₃)₂.

13. A method of selectively binding and releasing a molecule of interest, comprising:
a) providing a solid substrate coated with or covalently linked to an isomerizable organic molecule in a first isomer configuration;
b) contacting the coated solid substrate with a solution comprising a molecule of interest coupled to an isomer-specific affinity agent under a first condition to form a complex between the affinity agent and the isomerizable organic molecule coated on the solid substrate;
c) washing the complex formed in step (b) to remove matter not associated with the complex; and
d) converting the isomerizable organic molecule in the first isomer configuration to a second or subsequent isomer configuration under a second or subsequent condition (e.g., by applying a suitable isomerization-inducing agent), whereby the conversion of the organic molecule to the second or subsequent isomer configuration allows the molecule of interest to dissociate from the isomerizable organic molecule.

14. The method of claim 13, wherein the isomerizable organic molecule and the isomer-specific affinity agent together are the binding pair.

15. (canceled)

16. The method of claim 13, wherein the suitable isomerization-inducing agent is a light source configured to apply a specific or broad wavelength of light, such as a light-emitting diode or a lamp or wherein the suitable isomerization-inducing agent is a heat source or a cooling source.

17. (canceled)

18. The method of claim 13, wherein the method is performed to purify, separate, isolate or pattern the molecule of interest.

19. The method of claim 13, wherein the method further comprises coating or covalently linking the solid substrate with the isomerizable organic molecule in the first isomer configuration prior to step (a).

20. The method of claim 13, wherein the isomerizable organic molecule in the first isomer configuration is a compound of formula (IV-1a) or (V-1a): 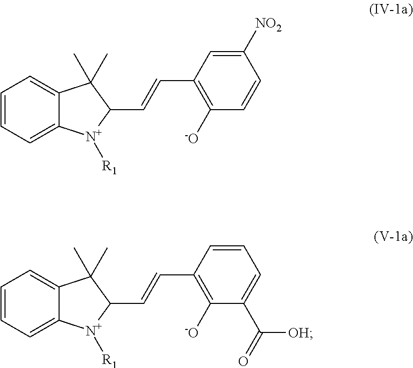
and
the isomerizable organic molecule in the second or subsequent isomer configuration is a compound of formula (IV-1) or (V-1): 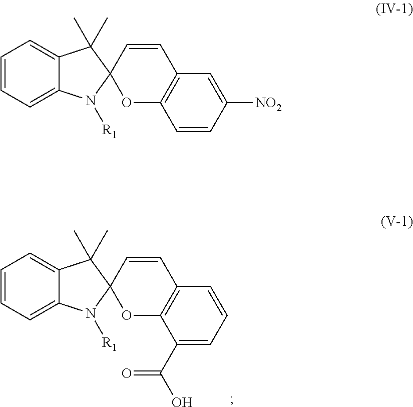
wherein R₁ is selected from hydrogen, (C₁-C₂₀) alkyl or substituted alkyl, (C₂-C₂₀) alkenyl, (C₂-C₂₀) alkynyl, (C₆-C₁₀) aryl, (5-12 atom) heteroaryl, (C₁-C₂₀) alkoxy, amino, halogen, cyano, trichloromethyl, trifluoromethyl, nitro, maleimido, sulfur, —C(O)—OH, azido or HO—(O)C—(C₁-C₃)alkyl.

21. The method of claim 13, wherein the solid substrate is placed in a housing, and washing from step (c) further comprises washing a solution through the housing to remove matter not associated with the solid substrate complex.

22. The method of claim 13, wherein the molecule of interest is a protein, an antibody, a drug, nucleic acid, a polymer, an organic molecule, a metal ion or an peptide.

23. The method of claim 13, wherein the isomer-specific affinity agent comprises a peptide aptamer selected from: LAREPTS (SEQ ID NO: 1), HELVRSP (SEQ ID NO: 2), WALDRGA (SEQ ID NO: 3), APSTPTP (SEQ ID NO: 4), SMPQTAG (SEQ ID NO: 5), EPLQLKM (SEQ ID NO: 6), ATPLWLK (SEQ ID NO: 7), AKIDART (SEQ ID NO: 8), YNHQRPP (SEQ ID NO: 9), ALIPKPR (SEQ ID NO: 10), QLITKPL (SEQ ID NO: 11), STPIQQP (SEQ ID NO: 12), TFAKSAY (SEQ ID NO: 13), STFTKSP (SEQ ID NO: 14), TPAHNDY(SEQ ID NO: 15), SLSLIQT (SEQ ID NO: 16), NQDVPLF (SEQ ID NO: 17), GPHLGLK (SEQ ID NO: 18), LAGPQMH (SEQ ID NO: 19), SQASSLK (SEQ ID NO: 20), SDLSSPY (SEQ ID NO: 21), LVTTWPA (SEQ ID NO: 22), ALYKNTS (SEQ ID NO: 23), QQISTQM (SEQ ID NO: 24), AWLPAGA (SEQ ID NO: 25), GTWLSRG (SEQ ID NO: 26), GHQVSRL (SEQ ID NO: 27), GPMLARG (SEQ ID NO: 28), TASLVRP (SEQ ID NO: 29), TDSLRLL (SEQ ID NO: 30), AKPDWRF (SEQ ID NO: 31), VQTYARV (SEQ ID NO: 32), YTEPVRE (SEQ ID NO: 33), MSLQQEH (SEQ ID NO: 34), MKWQTV (SEQ ID NO: 35), NERALTL (SEQ ID NO: 36), SPSTHWK (SEQ ID NO: 37), WSTTNVP (SEQ ID NO: 38), YHKLDPL (SEQ ID NO: 39), LSNNNLR (SEQ ID NO: 40), LPAGRVL (SEQ ID NO: 41), STSFWIT (SEQ ID NO: 42), LGSPMSN (SEQ ID NO: 43), WELRPRT (SEQ ID NO: 44), TNIESLK (SEQ ID NO: 45), LYAEVIR (SEQ ID NO: 46), NHSTQMY (SEQ ID NO: 47), NLQIYAV (SEQ ID NO: 48), YDTSSAS (SEQ ID NO: 49), MHSNTWD (SEQ ID NO: 50), TPTTVSY (SEQ ID NO: 51), TSHSILQ (SEQ ID NO: 52), TLRVPPNPNMNV (SEQ ID NO: 53), HVKLVAVADLMN (SEQ ID NO: 54), YHPNGMNPYTKA (SEQ ID NO: 55), EYSPKLFPPHRL (SEQ ID NO: 56), HHLTHANSLTNT (SEQ ID NO: 57), WHWGLLYPASAN (SEQ ID NO: 58), KPLMTYKVIHYV (SEQ ID NO: 59), QPDLSHPSTNAY (SEQ ID NO: 60), FPTNLATRSAMV (SEQ ID NO: 61), LAGPQMHGK (SEQ ID NO: 62), PLRPKSEYPFHY (SEQ ID NO: 63), SYSPKLFPPHRL (SEQ ID NO: 64), SDLSSPYG (SEQ ID NO: 65), SDLSSPYGG (SEQ ID NO: 66), SDLSSPYGGC (SEQ ID NO: 67) or EYSPKLFPPHRLGK (SEQ ID NO:68), or any one of SEQ ID NOs 1 to 67 wherein a glycine is appended to the C-terminus, or any one of SEQ ID NOs 1 to 67 wherein one or more amino acids are appended to either the N-terminus or the C-terminus.

24. (canceled)

25. A chromatographic apparatus for selectively binding and releasing a molecule of interest, the apparatus comprising:
a housing;
a coated solid substrate packed within the housing, the coating being a photoisomerizable organic molecule in a first isomer configuration, a second or subsequent isomer configuration, or a combination thereof;
at least one light source of suitable wavelength to convert the photoisomerizable organic molecule in the first isomer configuration to a second or subsequent isomer configuration,
wherein the coated solid substrate is in light contact with the at least one light source.

26.-27. (canceled)

28. The apparatus of claim 25, wherein the photoisomerizable organic molecule in the first isomer configuration is a compound of formula (IV-1a) or (V-1a): 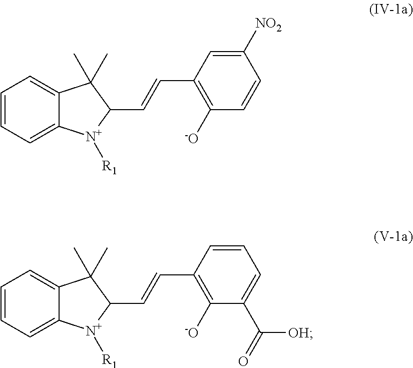
and
the photoisomerizable organic molecule in the second or subsequent isomer configuration is a compound of formula (IV-1) or (V-1): 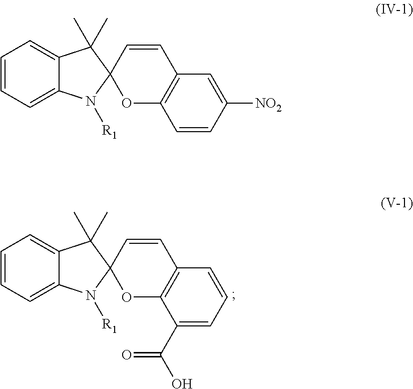
wherein R₁ is selected from hydrogen, (C₁-C₂₀) alkyl or substituted alkyl, (C₂-C₂₀) alkenyl, (C₂-C₂₀) alkynyl, (C₆-C₁₀) aryl, (5-12 atom) heteroaryl, (C₁-C₂₀) alkoxy, amino, halogen, cyano, trichloromethyl, trifluoromethyl, nitro, maleimido, sulfur, —C(O)—OH, azido or HO—(O)C—(C₁-C₃)alkyl.

29. A kit for selectively binding a molecule of interest, comprising:
the binding pair of claim 1;
instructions for binding the affinity agent to a molecule of interest and subsequently selectively binding to and optionally releasing the affinity agent from the isomerizable organic molecule; and
the kit optionally comprises a light source of a broad or specific wavelength suitable to convert the isomerizable organic molecule in the first isomer configuration to a second or subsequent configuration.

30. (canceled)
